(19) United States
(12) Patent Application Publication
(10) Pub. No.: US 2022/0411681 A1
(43) Pub. Date: Dec. 29, 2022

(54) Adhesive Composition

(71) Applicant: CAMBRIDGE UNIVERSITY HOSPITALS NHS FOUNDATION TRUST, Cambridge (GB)

(72) Inventors: Andrew Hindmarsh, Cambridge (GB); Alex Skordos, Cambridge (GB); Nandita Rahatekar, Cambridge (GB); Vijay Kumar Thakur, Cambridge (GB)

(21) Appl. No.: 17/781,314

(22) PCT Filed: Nov. 30, 2020
(86) PCT No.: PCT/GB2020/053068

§ 371 Date: May 31, 2022

(30) Foreign Application Priority Data
Nov. 28, 2019 (GB) 1917385.5

(51) Int. Cl. C09J 167/04; C09J 4/06; A61L 31/10; A61L 24/04

(52) U.S. Cl. CPC C09J 167/04; C09J 4/06; A61L 31/10; A61L 24/043; A61L 2430/34

(57) ABSTRACT

An adhesive composition comprising a cyanoacrylate and a solidifying polymer. The composition may further comprise a hydrophilic material, e.g. silica particles, and/or one or more polymerisation inhibitors. The adhesive composition may be disposed on a surgical mesh. The surgical mesh carrying the adhesive composition may be used for hernia repair.

### FIELD OF THE INVENTION

The present invention relates to adhesive compositions, in particular adhesive compositions for surgical applications, and surgical meshes carrying said adhesive compositions.

### BACKGROUND

Use of a mesh in hernia repair is known. Known fixing means to hold the mesh in place against a subject's tissue include sutures, and dissolvable barbs disposed on the surface of the mesh. However, such mechanical fixings may cause the subject pain.

U.S. Pat. No. 6,797,107 discloses a solid cyanoacrylate adhesive composition which polymerises into an adhesive polymer upon liquefying.

US 2015/0157439 discloses attachment of surgical meshes to tissue by a laser tissue soldering process using a biological polymer solder.

It is an object of the invention to provide an improved adhesive composition suitable for use in surgical applications.

### SUMMARY OF THE INVENTION

The present inventors have unexpectedly found that the adhesion of certain adhesive compositions to a surface may be enhanced by including a hydrophilic material in the composition.

Accordingly, in a first aspect there is provided an adhesive composition comprising a cyanoacrylate; a solidifying polymer; and a hydrophilic material.

Optionally, the hydrophilic material is a particulate hydrophilic material, optionally hydrophilic silica.

Optionally, the adhesive composition further comprises a polymerisation inhibitor.

The present inventors have surprisingly found that certain adhesive compositions containing more than one polymerisation inhibitor may increase the storage lifetime of the glue composition as compared to an adhesive composition containing one polymerisation inhibitor, or fewer polymerisation inhibitors.

Accordingly, in a second aspect there is provided an adhesive composition comprising a cyanoacrylate; a solidifying polymer; and at least two polymerisation inhibitors.

Optionally according to the second aspect, a first of the polymerisation inhibitors is a carboxylic acid.

Optionally according to the second aspect, a second of the polymerisation inhibitors is a hydroquinone.

Optionally according to the second aspect, the cyanoacrylate is an alkyl cyanoacrylate.

Optionally according to the second aspect, the alkyl cyanoacrylate is octyl cyanoacrylate.

In a third aspect, there is provided an adhesive composition comprising a cyanoacrylate and a solidifying polymer disposed on a surface of a surgical mesh.

Optionally according to the third aspect, the adhesive composition further comprises a hydrophilic material.

Optionally according to the third aspect, the adhesive composition further comprises at least one polymerisation inhibitor.

Optionally according to the third aspect, the adhesive composition comprises at least two different polymerisation inhibitors.

Optionally according to the third aspect, the adhesive composition covers substantially all of the surface of the surgical mesh.

Optionally according to the third aspect, the surgical mesh is disposed in an airtight packaging.

In a fourth aspect, there is provided a medical device, optionally an implantable medical device, having an adhesive or a surgical mesh according to any one of the first to third aspects disposed on a surface thereof.

In a fifth aspect, there is provided a method comprising applying the adhesive composition according to the third aspect onto the surface of the surgical mesh.

Optionally according to the fifth aspect, the adhesive composition is applied with a roller.

Optionally according to the fifth aspect, the adhesive composition is applied onto the surgical mesh in an anhydrous atmosphere.

Optionally according to the fifth aspect, the adhesive composition is applied to substantially all of the surface of the surgical mesh.

In a sixth aspect, there is provided a method of surgery comprising adhering an adhesive according to the first or second aspect, or a surgical mesh according to the third aspect, to tissue of a human or animal subject.

Optionally according to the sixth aspect the surgery is a hernia repair, optionally an abdominal wall, groin or diaphragmatic hernia.

Optionally according to the sixth aspect the adhesive composition or the surgical mesh is disposed on a surface of a medical device.

Optionally, according to the sixth aspect, the medical device is an implantable medical device which is implanted into a cavity of the human or animal subject.

### DESCRIPTION OF THE DRAWINGS

The present invention is described in conjunction with the appended figures. It is emphasized that, in accordance with the standard practice in the industry, various features are not drawn to scale. In fact, the dimensions of the various features may be arbitrarily increased or reduced for clarity of discussion.

FIG. 1 illustrates a surgical mesh according to some embodiments of the present invention;

FIGS. 2A-2D are scanning electron micrographs of an adhesive composition according to some embodiments which does not contain silica at, respectively, 50, 120, 500 and 5,000× magnification;

FIG. 3A-3D are scanning electron micrographs of an adhesive composition according to some embodiments which contains silica at, respectively, 50, 120, 500 and 5,000× magnification;

### DETAILED DESCRIPTION

The adhesive composition as described herein contains at least one cyanoacrylate and a solidifying polymer.

The cyanoacrylate may have formula CN—C(═CH₂)—COOR wherein R is selected from linear, branched or cyclic C₁₋₂₀ alkyl wherein one or more non-adjacent C atoms may be replaced with O; phenyl which may be unsubstituted or substituted with one or more C₁₋₁₂ alkyl groups; and alkylphenyl.

A preferred cyanoacrylate is octyl cyanoacrylate.

The cyanoacrylate preferably forms at least 50% by weight of the adhesive composition, optionally 50-90 weight % of the composition.

The solidifying polymer, and the adhesive composition containing the solidifying polymer as described herein, is preferably solid at 10° C., and optionally at 20° C. The adhesive composition is preferably a liquid at 37° C.

The solidifying polymer is preferably a homopolymer or copolymer comprising caprolactone repeat units, preferably poly(ε-caprolactone).

The polystyrene equivalent weight average molecular weight (Mw) of the solidifying polymer may be in the range of about 5,000-100,000.

The solidifying polymer optionally makes up 10-45 weight % of the adhesive composition, optionally 20-40 weight %.

The cyanoacrylate:solidifying polymer weight ratio is optionally in the range of 1:0.1-1:0.75, optionally 1:0.3-1:0.7.

The adhesive composition may contain a hydrophilic material. The hydrophilic material may be present in the composition in the form of a particulate hydrophilic material.

A polymerised film of the composition containing the hydrophilic material has a lower contact angle with deionised water at 20° C., optionally at least 5° lower, as compared to a polymerised film of a composition in which the hydrophilic material is absent. The contact angle may be as measured by the sessile drop technique using a Biolin Scientific Theta Lite Attension Tensiometer.

The hydrophilic material may be a hydrophilic silica, optionally hydrophilic fumed silica. Hydrophilic silica preferably has silanol (Si—OH) groups at the surface thereof.

Hydrophilic particles as described herein, e.g. hydrophilic silica particles, optionally have an average particle (aggregate) length in the range of about 0.1-0.4 μm, optionally of 0.2-0.3 μm. Hydrophilic silica particles as described herein optionally have a BET surface area of 50-500 m²/g

In use, the hydrophilic material may draw a greater amount of water into the composition as compared to an adhesive composition in which the hydrophilic material is not present. Water in the composition may facilitate activation of the cyanoacrylate. Without wishing to be bound by any theory, drawing water into the composition may enhance activation of the cyanoacrylate.

The hydrophilic material optionally provided in an amount of 0.1-10%, optionally 1-5 weight % of solidifying polymer+cyanoacrylate weight.

The adhesive composition may contain one or more polymerisation inhibitors selected from free radical and ionic polymerisation inhibitors. Exemplary polymerisation inhibitors include, without limitation, hydroquinone; and C1-25 alkanoic acids. Preferred alkanoic acids are C2-20 alkanoic acids, more preferably acetic acid and stearic acid.

The, or each, polymerisation inhibitor which is a solid at 20° C. may be present in the adhesive composition in an amount in the range of 0.1-2 weight %.

The, or each, polymerisation inhibitor which is a liquid at 20° C., e.g. acetic acid, may be present in the adhesive composition in an amount in the range of 0.1-1 ml/g. Optionally, at least some of the liquid inhibitor evaporates upon or following formation of a film of the composition and/or during polymerisation of the film.

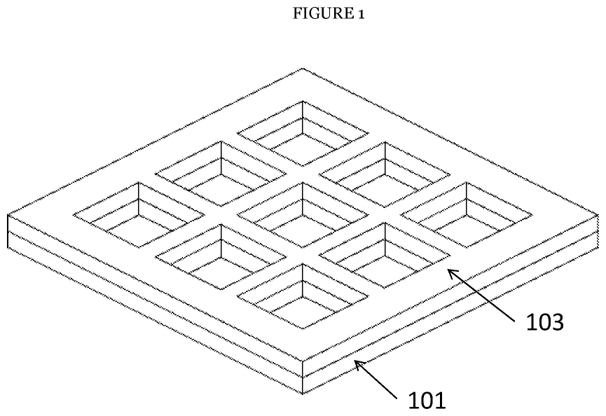

FIG. 1 illustrates a surgical mesh 101 having a layer of an adhesive composition 103 disposed on a surface thereof according to some embodiments.

In FIG. 1, the layer of adhesive composition 103 does not cover the apertures in the mesh. In other embodiments, it will be appreciated that some or all apertures may be covered by the adhesive composition and/or the adhesive composition may be disposed in some or all of the apertures of the mesh.

The mesh may be formed from any suitable mesh material known for use in surgery including, without limitation, synthetic polymers, biopolymers and combinations thereof. Synthetic polymers include, without limitation, polypropylene (PP), polyethylene terephthalate (PET); polytetrafluoroethene (PTFE), polyglycolic acid and combinations thereof. An exemplary biopolymer is collagen. A polymer mesh may be at least partially covered with a metal or oxide thereof, e.g. titanium or titanium oxide.

Optionally, the apertures in the mesh have an area in the range of about 0.1-5 mm, preferably about 0.5-2 mm.

Optionally, the mesh has a width and/or length in the range of up to about 30 cm, optionally up to about 20 cm.

It will be understood that the mesh is flexible, allowing it to conform to any contours of a surface to which it is applied.

The adhesive composition may form a continuous layer covering substantially the entire surface of the mesh (e.g. at least 90% or at least 95% of the surface area of the mesh), as illustrated in FIG. 1. In other embodiments, a plurality of non-contiguous islands of adhesive may be provided at different points on the surface of the mesh.

The adhesive composition may be applied to the mesh using any known application technique. A preferred coating technique is roll-coating. Roll coating is suitable for forming a continuous layer of the adhesive composition. The surface of the roller used in roll coating may have a low adhesion to the adhesive composition, e.g. PTFE.

The adhesive composition may be applied onto the mesh in a roll-to-roll process. Following coating of a feed roll and prior to rolling up of the coated roll, a non-stick sheet may be applied to the coated surface.

A technique for point application of the adhesive composition is by use of an applicator such as a glue gun.

The adhesive may be applied in a low moisture environment (e.g. less than 5% humidity at 20° C.). An exemplary low moisture environment is a nitrogen or argon atmosphere, or a dry room containing dried atmospheric air.

The adhesive-coated mesh may be sealed in airtight packaging, e.g. a vacuum package. The airtight packaging may have two or more layers of material. The airtight packaging may have an embossed inner and/or outer surface.

One or both surfaces of the adhesive-coated mesh may carry a non-stick sheet, e.g. parchment paper or a plastic film, to prevent adhesion of the mesh to the interior of the airtight packaging.

The adhesive-coated mesh may be stored at below 10° C., preferably at no more than 5° C., until ready for use.

In use, the adhesive coated mesh may be removed from any packaging and applied to tissue requiring adhesion for repair, e.g. a hernia repair.

Heat of up to 50° C. may be applied to effect liquefaction of the adhesive composition and binding to the tissue. The composition may be heated by any suitable technique including, without limitation, contacting the mesh with a heated object, passing an electrical current through the mesh or placing the mesh on a heater. The adhesive coated mesh may be used in, without limitation, laparoscopic surgery or open surgery.

Although the invention has been described herein with reference to an adhesive composition disposed on a mesh, it will be understood that the adhesive composition may be used in other surgical or non-surgical applications.

In some embodiments, the adhesive composition is disposed on a mesh and the mesh is applied to hold tissue together to prevent a hernia, i.e. to prevent an internal part of the body from pushing through the tissue. It will be understood that the tissue in these embodiments may or may not be damaged.

In some embodiments, the adhesive composition may be used to re-join two surfaces of a subject's tissue, e.g. two surfaces of a tissue which have been separated by a wound or a surgical incision. The adhesive composition according to these embodiments may be applied directly between the two separated surfaces without a mesh, or across the two separated surfaces with a mesh.

In some embodiments, the adhesive composition may be used to attach an implantable medical device to a subject's internal tissue, e.g. within a subject's abdominal cavity. In some embodiments, the medical device carries the adhesive on a surface thereof. In some embodiments, the medical device is attached to a mesh having the adhesive composition disposed on a surface of the mesh. A protective backing layer may cover the adhesive layer during storage of the medical device. Medical devices as described herein include, without limitation, devices configured to monitoring a physiological parameter and devices configured to release a medicament. Medical devices as described herein are optionally Class III devices under USC 21 CFR860 or Class III devices under Article IX of European Council Directive 93/42/EEC.

### EXAMPLES

### Adhesive Composition Examples

Adhesive compositions were prepared as set out in Table 1.

| | | Poly(e- | | | |
|---|---|---|---|---|---|
| | | caprolactone) | OCA:PCL | Fumed | |
| Composition | OCA | 50,000 Mw | weight | silica | |
| Example | (wt %) | (wt %) | ratio | (wt %) | Polymerisation inhibitors |
| 1 | 66.7 | 33.3 | 1:0.5 | 2 | Hydroquinone (2 weight %) |
| | | | | | Steric acid (1 weight %) |
| | | | | | Acetic acid (0.5 ml/g) |
| 2 | 66.7 | 33.3 | 1:0.5 | 0 | Hydroquinone (2 weight %) |
| | | | | | Steric acid (1 weight %) |
| | | | | | Acetic acid (0.5 ml/g |

OCA: 2-octyl cyanoacrylate: Polycaprolactone (50k MW)
PCL: poly(ε-caprolactone) 50,000 Mw

In Table 1, weight percentage values for fumed silica and polymerisation inhibitors are based on the combined weight of OCA and PCL alone.

In Table 1, ml/g values for acetic acid are based on the weight of OCA in the composition.

Films of Composition Examples 1 and 2 were formed by blade coating onto a substrate and polymerised.

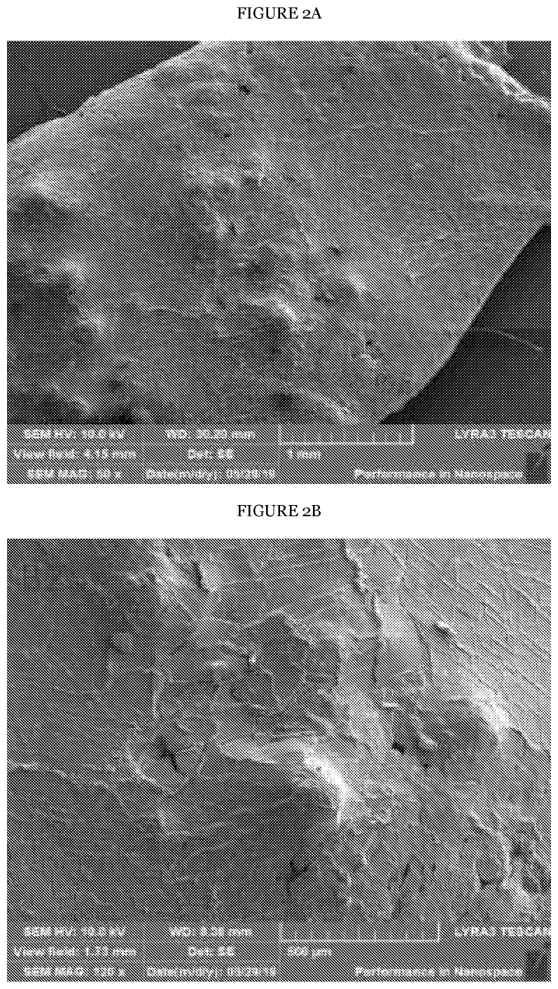

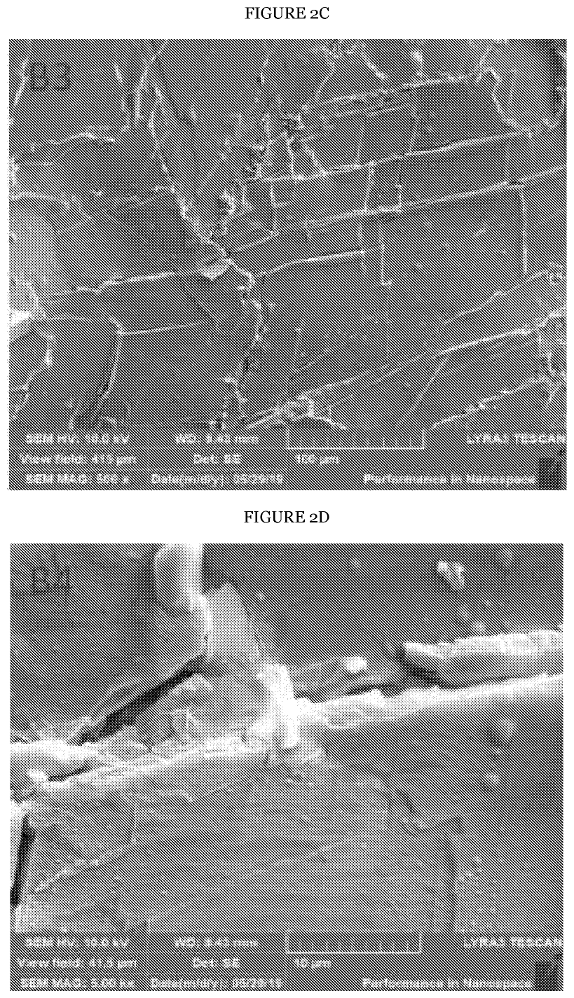

With reference to FIGS. 2A-2D, a uniform film was formed following polymerisation of Composition Example 2, containing no silica.

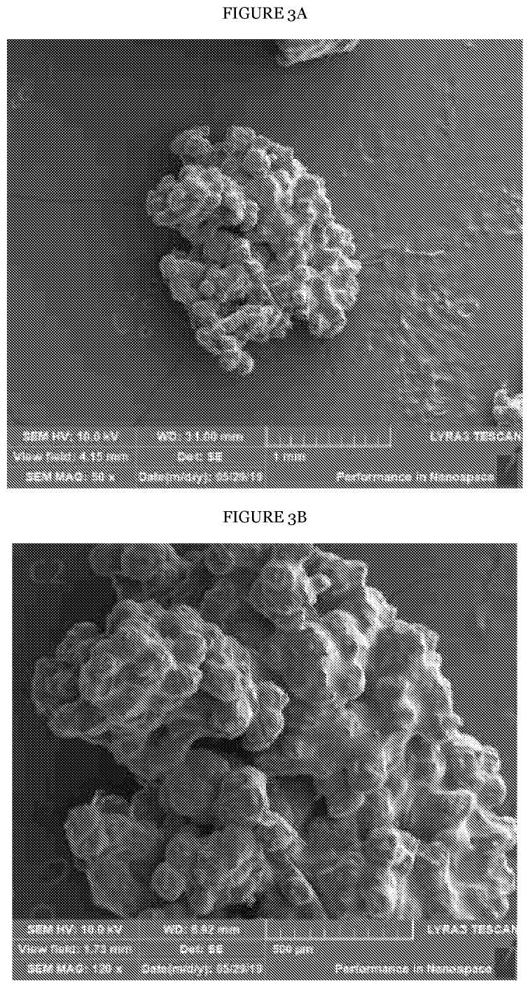

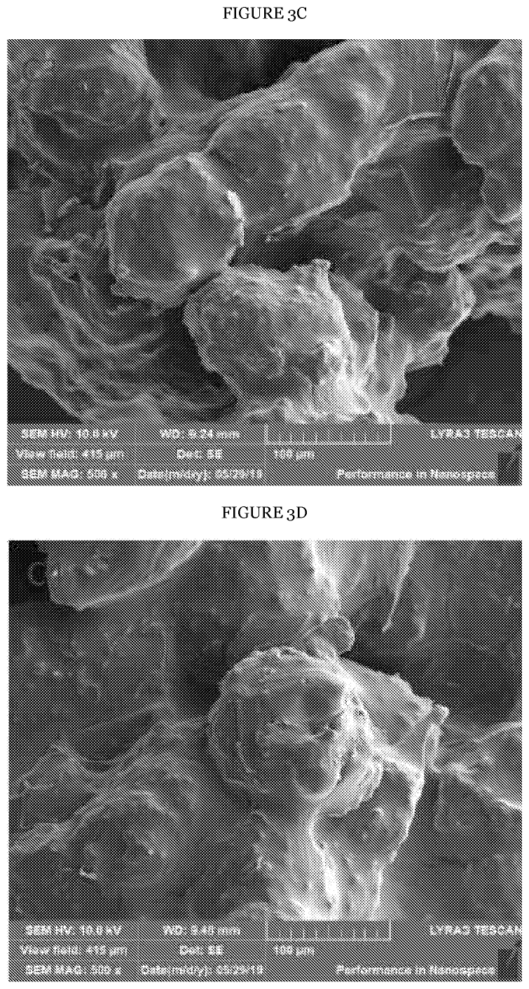

With reference to FIGS. 3A-3D, a uniform film was formed following polymerisation of Composition Example 1, with the additional presence of uniformly distributed clumps of silica.

Contact Angle Measurement

A film of OCA and fumed silica in a 1:2 OCA: silica ratio was formed on a PTFE substrate and polymerised at room temperature for 3 days.

The contact angle of deionised water was measured by the sessile drop technique using a Biolin Scientific Theta Lite Attension Tensiometer. The film was examined on 2-3 different areas and contact angle was calculated as an average of the left- and right-angle of liquid drop.

The contact angle was 55.6±2.6°

For comparison, the contact angle for a film of OCA prepared in the same way was 75.6±2.2°.

Adhesive Mesh

TiMESH surgical mesh obtained from pfm medical of titanised type 1a polypropylene meshes having a macroporous pore size of 1 mm was cut to a square of 5 cm×5 cm and placed on a first sheet of parchment paper (8 cm×8 cm) in a 700 nm deep trough formed within a polypropylene square and placed in an anhydrous (2 weight % water) argon environment.

Adhesive Composition 1 was applied to the surface of the mesh and a second sheet of parchment paper was placed over the mesh and adhesive composition. The adhesive composition was spread using a PTFE roller applied to the second sheet of parchment paper at ambient temperature to give a 200 micron layer of the adhesive composition on the mesh.

The first and second sheets of parchment paper were removed and the surgical mesh was placed in a vacuum bag having an embossed, air impermeable polyamide exterior and a polyethylene interior. The bag was evacuated and heat-sealed using an iLmyh Automatic Food Vacuum Sealing Machine.

Adhesion Testing of Material on Dry and Wet Balsa Wood Substrate

Balsa wood adherents of a thickness of 2 mm were cut to dimensions of 10 mm×30 mm and in the case of wet testing dipped in water for five minutes prior to the experiment. An overlap length of 15 mm was used between the two adherents in a single lap shear arrangement. Once the overlap region of one of the balsa wood pieces had been coated with freshly formulated adhesive using a blade, the two wood pieces were maintained in contact under a pressure of 19 kPa. The specimens were then kept in an oven at 37° C. for adhesive polymerisation for 10 and 30 minutes. Tests were carried out in an Instron universal testing machine in which adhered wood pieces were loaded at a speed of 2 mm/min. Two specimens were tested per case. The average shear lap strength was 312 kPa and 125 kPa for Composition 2 for dry and wet conditions respectively; and 50 kPa and 127 kPas for Composition 1 (formulation containing silica) for dry and wet conditions respectively. This shows that the strength of the silica containing adhesive increases in a wet environment in contrast to the non silica containing adhesive.

Adhesion Testing of Material Incorporated on Mesh on Wet Balsa Wood Substrate

Tests under conditions identical to the previous paragraph were carried out with the addition of the surgical mesh in the layer of adhesive coated to one of the adherent surfaces. Five specimens were tested per case. The results for wet balsa wood adherents showed a strength of 652±125 kPa for the silica containing formulation (Composition 1) and 786±148 kPa for the formulation without silica (Composition 2). This shows an equivalence of the two formulations under wet conditions when the adhesive is freshly produced.

Adhesion Testing of Material on Porcine Tissue

The packed adhesive mesh was stored in a refrigerator at 4° C. for 2 weeks, after which time the product was used to join two pieces of porcine tissue. The mesh was removed from its packing and applied across a join between the two pieces of porcine tissue. A blunt knife and a 1.4 kg metal block were heated to 50° C. in an oven. The flat of the knife blade was pressed against the surface of the mesh, and the heated metal block was then placed on the mesh. After some time to allow the mesh to adhere to the porcine tissue, the block was removed and the mesh was found to have bound well to the surface of the porcine tissue, thereby joining the two pieces of tissue together.

The same process was repeated using adhesive meshes which were sealed but not refrigerated, and stored in either air or in an argon chamber. No adhesion was achieved with the adhesive mesh stored in air without refrigeration. Some adhesion was achieved with the adhesive mesh stored in the argon chamber, but not to the same degree as the refrigerated mesh. Without wishing to be bound by any theory, acetic acid evaporation from the mesh stored at room temperature allows for a greater degree of polymerisation of the cyanoacrylate during storage as compared to the refrigerated mesh.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. An adhesive composition comprising a cyanoacrylate; a solidifying polymer; and a hydrophilic material.

2. An adhesive composition according to claim 1 wherein the hydrophilic material is a particulate hydrophilic material.

3. An adhesive composition according to claim 2 wherein particulate hydrophilic material is hydrophilic silica.

4. An adhesive composition according to claim 1 wherein the adhesive composition further comprises a polymerisation inhibitor.

5. An adhesive composition comprising a cyanoacrylate; a solidifying polymer; and at least two different polymerisation inhibitors.

6. An adhesive composition according to claim 5 wherein a first of the polymerisation inhibitors is a carboxylic acid.

7. An adhesive composition according to claim 5 wherein a second of the polymerisation inhibitors is a hydroquinone.

8. An adhesive composition according to claim 1 wherein the cyanoacrylate is an alkyl cyanoacrylate.

9. An adhesive composition according to claim 8 wherein the alkyl cyanoacrylate is octyl cyanoacrylate.

10. A surgical mesh comprising an adhesive composition disposed on a surface thereof, wherein the adhesive composition comprises a cyanoacrylate and a solidifying polymer.

11. A surgical mesh according to claim 10 wherein the adhesive composition further comprises a hydrophilic material.

12. A surgical mesh according to claim 10 wherein the adhesive composition further comprises at least one polymerisation inhibitor.

13. A surgical mesh according to claim 12 wherein the adhesive composition comprises at least two different polymerisation inhibitors.

14. The surgical mesh according to claim 10 wherein the adhesive composition covers substantially all of the surface of the surgical mesh.

15. A surgical mesh according to claim 10 disposed in an airtight packaging.

16. A medical device having an adhesive or a surgical mesh according to claim 10 disposed on a surface thereof.

17. A method comprising applying the adhesive composition according to claim 1 onto a surface of a surgical mesh.

18-20. (canceled)

21. A method of surgery comprising adhering an adhesive composition according to claim 1 to tissue of a human or animal subject.

22. The method according to claim 21 wherein the surgery is a hernia repair.

23. (canceled)

24. The method according to claim 23 wherein the adhesive composition is disposed on a surface of a medical device.

25. (canceled)
